(19) United States
(12) Patent Application Publication
(10) Pub. No.: US 2024/0206847 A1
(43) Pub. Date: Jun. 27, 2024

(54) ULTRASONIC MOTOR, ULTRASONIC PROBE, AND ULTRASONIC ENDOSCOPE SYSTEM

(71) Applicant: OLYMPUS MEDICAL SYSTEMS CORP., Tokyo (JP)

(72) Inventors: Yuichi SHIGA, Tokyo (JP); Tomoyuki KUGO, Matsudo-shi (JP); Takahiro KOYAMA, Tokyo (JP)

(73) Assignee: OLYMPUS MEDICAL SYSTEMS CORP., Tokyo (JP)

(21) Appl. No.: 18/597,318

(22) Filed: Mar. 6, 2024

Related U.S. Application Data
(63) Continuation of application No. PCT/JP2021/032656, filed on Sep. 6, 2021.

(51) Int. Cl. A61B 8/12; A61B 8/00

(52) U.S. Cl. CPC A61B 8/12; A61B 8/4461; A61B 8/4483; A61B 8/461

(57) ABSTRACT

An ultrasonic motor of the invention includes: a transducer including a plurality of piezoelectric elements and an elastic body having an outer wall surface on which the plurality of piezoelectric elements are secured, the transducer being configured to vibrate when an alternating voltage is applied to the plurality of piezoelectric elements; a rotator configured to rotate by receiving the vibration of the transducer by being pressed in contact with a surface of the transducer; a biasing spring configured to press the rotator against the elastic body; and an output shaft configured to output rotation of the rotator. The elastic body includes a hollow portion and houses the rotator and the biasing spring in the hollow portion, and the rotator rotates in a state of being pressed against an inner wall surface of the hollow portion of the elastic body by the biasing spring.

### CROSS-REFERENCE TO RELATED APPLICATION

This application is a continuation application of PCT/JP2021/032656 filed on Sep. 6, 2021, the entire contents of which are incorporated herein by this reference.

### BACKGROUND OF THE INVENTION

### 1. Field of the Invention

The present invention relates to an ultrasonic motor, an ultrasonic probe using the ultrasonic motor, and an ultrasonic endoscope system including the ultrasonic probe.

### 2. Description of the Related Art

Conventionally, ultrasonic endoscope systems, such as ultrasonically-guided puncture systems, have been put into practical use, and such systems are used to puncture a puncture needle into a lesion part as needed while searching for and observing the lesion part based on an ultrasonic image, and to suck and collect living tissues, cells, and the like of the lesion part. The living tissues, cells, and the like thus collected are utilized for a pathological examination and a pathological diagnosis.

Such type of the ultrasonically-guided puncture systems include an ultrasonic probe configured to be inserted into a body cavity of a living body, for example, a very thin lumen such as a peripheral part of a lung, a prostate gland, and the like, to obtain an ultrasonic image, and the ultrasonic probe has a function to puncture a desired lesion part with a puncture needle.

In recent years, there has been a constant desire for miniaturization, for example, for the ultrasonic probe of this type, and an effort for miniaturization has been made while maintaining a performance such as an image quality of an ultrasonic image, or while achieving a high performance.

As conventional ultrasonic probes, what is called, electronic scanning type ultrasonic probes have been widely spread. In such electronic scanning type ultrasonic probes, a plurality of transducers are arranged in a fan shape, and electronic scanning is performed sequentially with each of the transducers. Since such electronic scanning type ultrasonic probes require a large number of wirings, there is a limitation in the diameter reduction of the ultrasonic probes. In addition, in accordance with the miniaturization of the ultrasonic probes, each of the transducers is miniaturized, which tends to degrade a quality such as an image quality. Thus, there has been a problem that there is a certain degree of limitation in the miniaturization of the electronic scanning type ultrasonic probe.

In order to achieve further miniaturization of an ultrasonic probe, it can be considered that a mechanical scanning type ultrasonic probe is employed, for example. In the mechanical scanning type ultrasonic probe, scanning is performed by causing a transducer (piezoelectric element) for ultrasonic image generation to oscillate mechanically. The ultrasonic probe of the mechanical scanning type can have only one wiring and one transducer. Therefore, even if the transducer is miniaturized in accordance with the miniaturization of the ultrasonic probe itself, it can be expected that the diameter reduction and the miniaturization of the ultrasonic probe can be achieved simultaneously while suppressing deterioration of the image quality.

Therefore, in order to achieve the miniaturization of the mechanical scanning type ultrasonic probe, it can be considered that an ultrasonic motor is applied, for example, as a driving actuator for oscillating the transducer.

As a configuration of a conventional ultrasonic motor, Japanese Patent Application Laid-Open Publication No. 5-227770, for example, discloses a traveling-wave type ultrasonic motor, which is configured to drive and rotate a rotator by causing the transducer to vibrate, with the rotator pressurized by a biasing member such as a spring being pressed against the transducer.

### SUMMARY OF THE INVENTION

An ultrasonic motor according to one aspect of the present invention includes: a transducer including a plurality of piezoelectric elements and an elastic body having an outer wall surface on which the plurality of piezoelectric elements are secured, the transducer being configured to vibrate when an alternating voltage is applied to the plurality of piezoelectric elements; a rotator configured to rotate by receiving the vibration of the transducer by being pressed in contact with a surface of the transducer; a biasing spring configured to press the rotator against the elastic body; and an output shaft configured to output rotation of the rotator. The elastic body includes a hollow portion, and configured to house the rotator and the biasing spring in the hollow portion, and the rotator rotates in a state of being pressed against an inner wall surface of the hollow portion of the elastic body by the biasing spring.

An ultrasonic probe according to one aspect of the present invention includes the ultrasonic motor, and an ultrasonic transducer configured to receive rotation of the output shaft, to transmit and receive an ultrasound.

An ultrasonic endoscope system according to one aspect of the present invention includes: an endoscope including the ultrasonic probe in a distal end of the endoscope; a processor; and a monitor. The ultrasonic probe is configured to transmit an ultrasound to a subject, and receive the ultrasound reflected by the subject, the processor is configured to generate an image signal based on the ultrasound received by the ultrasonic probe, and the monitor is configured to receive the image signal to display an image of the subject.

### BRIEF DESCRIPTION OF THE DRAWINGS

FIG. 1 is a perspective view showing an appearance of an ultrasonic motor according to a first embodiment of the present invention;

FIG. 2 is a vertical cross-sectional view cut along a plane which is parallel to a surface shown by the arrows [2] in FIG. 1 and which includes an axial center of an output shaft;

FIG. 3 is a cross-sectional perspective view showing a main part of an internal configuration of a distal end portion of an ultrasonic probe according to a second embodiment of the present invention; and

FIG. 4 is a schematic configuration view schematically showing an overall configuration of an ultrasonic endoscope system according to a third embodiment of the present invention.

### DETAILED DESCRIPTION OF THE PREFERRED EMBODIMENT(S)

For example, in a case where the ultrasonic motor having the configuration disclosed by the Japanese Patent Application Laid-Open Publication No. 5-227770 is miniaturized to be applied to an ultrasonic probe, a length of a transducer in a longitudinal axis direction becomes short, which causes a problem that a resonant frequency becomes high.

In general, there is such a problem that the higher a resonant frequency of a transducer, the greater an attenuation rate of an outputted ultrasonic vibration or the shorter a reach distance of the ultrasonic vibration. Therefore, it is known that a desirable configuration is such that the resonant frequency of an ultrasonic transducer is maintained as low as possible.

In addition, generally, an ultrasonic probe is configured such that a part around a transducer for ultrasonic image generation is filled with an ultrasonic transmission medium (for example, a liquid oil, a jelly oil, or the like). Therefore, when the ultrasonic motor having the configuration disclosed by the Japanese Patent Application Laid-Open Publication No. 5-227770 is applied as a driving actuator for oscillating the transducer for ultrasonic image generation, the following problems are pointed out.

In the ultrasonic motor disclosed by the Japanese Patent Application Laid-Open Publication No. 5-227770, the rotator and the transducer contact each other, to thereby cause the vibration of the transducer to be transmitted to the rotator. In this case, the part at which the rotator and the transducer contact each other (region including the operating point) is exposed outside.

In the case where the ultrasonic motor having such a configuration is applied to the ultrasonic probe, if the region including the operating point of the ultrasonic motor is arranged in the space filled with the ultrasonic transmission medium, there is a possibility that the frictional force of the contacting part of the ultrasonic motor is reduced remarkably. Such a remarkable reduction of the frictional force causes a problem that the ultrasonic motor cannot obtain a sufficient rotation torque. Therefore, it is difficult to employ the ultrasonic motor having the conventional configuration as it is as the driving source of the ultrasonic probe.

In view of the above, the present invention can provide: an ultrasonic motor capable of achieving miniaturization while ensuring a length of a transducer in a longitudinal axis direction to maintain a low resonant frequency, and also capable of obtaining a sufficient rotation torque; an ultrasonic probe using the ultrasonic motor; and an ultrasonic endoscope system including the ultrasonic probe.

Hereinafter, the present invention will be described by illustrated embodiments.

The respective drawings used in the following description are schematically shown, and in order to show each of the constituent elements in a size recognizable in the drawings, there is a case where the dimensional relationship, scales, etc., of the respective members are made different for each of the constituent elements. Therefore, the present invention is not limited only to the illustrated forms with regard to the number, the shapes, the ratio of the sizes, the relative positional relationship, etc., of the respective constituent elements illustrated in each of the drawings.

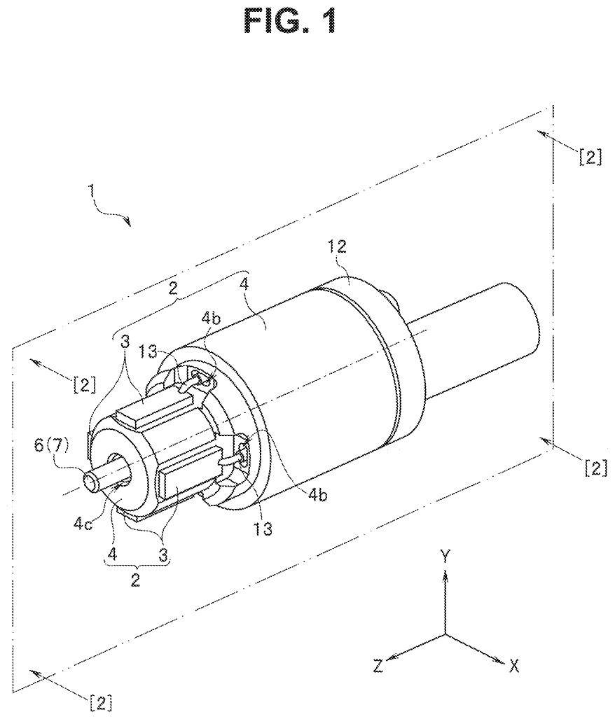

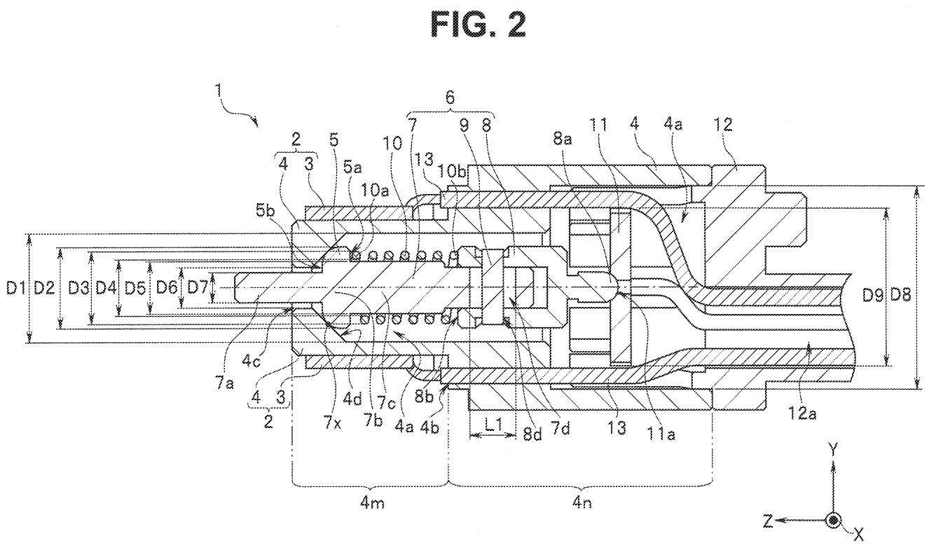

First, a configuration of an ultrasonic motor according to a first embodiment of the present invention will be described below with reference to FIGS. 1 and 2. FIG. 1 is a perspective view showing an appearance of the ultrasonic motor of the first embodiment of the present invention. FIG. 2 is a vertical cross-sectional view cut along a plane which is parallel to a surface shown by the arrows [2] in FIG. 1 and which includes an axial center of an output shaft.

Note that the direction shown by the arrow sign Z in FIGS. 1 and 2 is referred to as the direction along a rotational axis of the ultrasonic motor according to the present embodiment. In the following description, the direction shown by the arrow sign Z is referred to as a Z-axis direction. In this case, the left direction on the drawings is defined as the distal end side of the ultrasonic motor, and the right direction on the drawings is defined as the proximal end side of the ultrasonic motor. In addition, the directions shown by the arrow signs X, Y in FIGS. 1 and 2 respectively indicate the directions orthogonal to the Z-axis direction.

As shown in FIGS. 1 and 2, the ultrasonic motor 1 according to the present embodiment includes a transducer for driving the ultrasonic motor 2 (3, 4), a rotator 5, an output shaft 6 (7, 8, 9), a biasing member 10, a support board member 11, a rear cylindrical member 12, and a power supply cable 13.

The transducer for driving the ultrasonic motor 2 (hereinafter, simply referred to as the transducer 2) constitutes a stator of the ultrasonic motor 1. The transducer 2 is configured of a plurality of piezoelectric elements for driving 3 (hereinafter, simply referred to as piezoelectric elements 3) and a metallic elastic body for driving 4 (hereinafter, simply referred to as the elastic body 4). Here, the transducer 2 is configured such that the plurality of piezoelectric elements 3 are pasted (secured) at a predetermined position (outer wall surface) of the elastic body 4. The transducer 2 vibrates when an alternating voltage is applied to the plurality of piezoelectric elements 3.

The plurality of piezoelectric elements 3 are electromechanical conversion elements. As an example of the ultrasonic motor 1 of the present embodiment, an example in which four piezoelectric elements 3 are provided is shown. The power supply cable 13 is connected to each of the piezoelectric elements 3. The power supply cable 13 is a cable for power supply. The power supply cable 13 is introduced from outside of the ultrasonic motor 1 into an insertion path 12a of the rear cylindrical member 12, passed through a hollow portion 4a of the elastic body 4, to be led out to the outside from a through hole 4b provided near an outer peripheral edge portion of the elastic body 4, and connected to each of the piezoelectric elements 3.

With such a configuration, when the alternating voltage, with the phase difference being 90 degrees and the driving frequency being the resonant frequency of the elastic body 4, is applied to each of the four piezoelectric elements 3, the elastic body 4 is excited. Then, the surface of the elastic body 4 generates substantially a circular motion viewed from the cross section (detailed operation will be described later).

The elastic body 4 is formed in substantially a columnar shape, as a whole, and includes inside thereof the hollow portion 4a. The elastic body 4 is formed by processing a metal material or the like, for example.

In detail, as shown in FIG. 2, the elastic body 4 of the present embodiment includes a first region 4m and a second region 4n. The first region 4m is located at a part closer to the distal end (first end) and is formed in substantially a columnar shape whose outer diameter is small. The second region 4n is connected to the first region 4m to be integrated therewith, and formed in substantially a columnar shape whose outer diameter is larger than that of the first region 4m.

Note that the hollow portion 4a provided inside the first region 4m and the second region 4n is continuous to form a single space. In this case, the hollow portion 4a of the elastic body 4 is formed as a pillar-shaped space having a small diameter of an inner diameter D1, from the region corresponding to the first region 4m to the middle position of the region corresponding to the second region 4n. In addition, the hollow portion 4a is formed as a pillar-shaped space having a diameter (inner diameter D8) larger than the inner diameter D1, from the middle position of the region corresponding to the second region 4n to the proximal end side of the hollow portion 4a (D8>D1).

In the hollow portion 4a, the main part of the output shaft 6, the rotator 5, the biasing member 10, and the support board member 11 are disposed.

As described above, the power supply cable 13 passes through the hollow portion 4a. Note that the through hole 4b through which the power supply cable 13 is led out to the outside of the elastic body 4 is a stepped part created by a difference between the outer diameter of the first region 4m and the outer diameter of the second region 4n, and the though hole 4b opens toward the front face at a position near the outer peripheral edge portion of the second region 4n.

On the outer circumferential surface (outer wall surface) of the first region 4m of the elastic body 4, the four piezoelectric elements 3 are arranged in the circumferential direction at substantially equal intervals. The four piezoelectric elements 3 are pasted on the outer circumferential surface of the elastic body 4 using an adhesive or the like, for example.

On the surface which is located on the most distal end (first end) of the first region 4m of the elastic body 4, a through hole 4c penetrating through substantially the center of the surface on the most distal end is formed. The through hole 4c is a through hole for allowing the hollow portion 4a to communicate with outside. In the through hole 4c, a part of the output shaft 6 (to be detailed later; a first shaft end region 7a of a first rotation shaft 7) provided in the hollow portion 4a is rotatably disposed.

The elastic body 4 has a tapered surface 4d formed toward the through hole 4c. The tapered surface 4d is formed on the inner wall surface of the hollow portion 4a in the first region 4m of the elastic body 4. In other words, the tapered surface 4d formed in a tapered shape which expands toward the proximal end side, with the through hole 4c as a center.

The most proximal end side of the second region 4n of the elastic body 4 is formed as an opening. In this opening, the rear cylindrical member 12 is provided. With such a configuration, the rear cylindrical member 12 functions as a lid member closing the opening on the proximal end side of the elastic body 4. In addition, the rear cylindrical member 12 protects the hollow portion 4a from the proximal end side. The rear cylindrical member 12 includes the insertion path 12a penetrating therethrough. Such a configuration enables the power supply cable 13 to be introduced from outside into the hollow portion 4a through the insertion path 12a.

As described above, in the hollow portion 4a of the elastic body 4, the output shaft 6, the rotator 5, the biasing member 10, and the support board member 11 are disposed.

The rotator 5 constitutes a rotor of the ultrasonic motor 1. The rotator 5 receives a vibration generated by the transducer 2 by being pressed by a biasing force of the biasing member 10, with the rotator contacting a predetermined part of the surface of the elastic body 4 of the transducer 2, and thereby the rotator 5 rotates. Although detail will be described later, the rotator 5 in the ultrasonic motor 1 of the present embodiment is shown by taking the configuration in which the rotator 5 is formed integrally with the output shaft 6 as an example.

The output shaft 6 is a shaft member that outputs rotation of the rotator 5. The output shaft 6 includes two components, i.e., the first rotation shaft 7 and a second rotation shaft 8, and a coupling pin 9 for coupling these two components (7, 8). Although the detail will be described later, configuring the output shaft 6 by coupling the two components, i.e., the first rotation shaft 7 and the second rotation shaft 8 with each other is a measure in the configuration for arranging the biasing member 10 coaxially with the first rotation shaft 7. Description will be made below on the detailed configuration of the output shaft 6.

Among the constituent members of the output shaft 6, the first rotation shaft 7 includes a first shaft end region 7a, a rotator arranging region 7b, and a biasing member arranging region 7c, as shown in FIG. 2.

In the state where the output shaft 6 is incorporated into the ultrasonic motor 1, the first shaft end region 7a is arranged in the through hole 4c of the elastic body 4 and a part on the distal-most side of the first shaft end region 7a protrudes outside to be exposed. The first shaft end region 7a is a shaft part configured to output the rotation output generated by the ultrasonic motor 1 to the outside. Therefore, the first rotation shaft 7 is arranged, with the rotation axis of the ultrasonic motor 1 as a center.

The rotator arranging region 7b is a region at which the rotator 5 is integrally formed. As described above, the rotator 5 in the ultrasonic motor 1 of the present embodiment is shown by taking the configuration in which the rotator 5 is formed integrally and coaxially with the output shaft 6 as an example. In detail, in the ultrasonic motor 1 of the present embodiment, the rotator 5 is formed integrally with the first rotation shaft 7 of the plurality of constituent members constituting the output shaft 6, and located at a predetermined position coaxial with the first rotation shaft 7. For example, the rotator 5 is formed at a middle part of the first rotation shaft 7 in the Z-axis direction. In addition, the rotator 5 is formed in substantially a disk shape.

As in the above-described configuration example, the rotator 5 may be formed integrally with the first rotation shaft 7 as one member inseparable from the first rotation shaft 7, or as another configuration example different from the above-described configuration example, the rotator 5 and the first rotation shaft 7 may be formed respectively as separate members, and then may be integrated by assembling the rotator 5 to the first rotation shaft 7. Thus, forming the rotator 5 integrally with the first rotation shaft 7 should be interpreted as meaning that, when these constituent members are incorporated into the ultrasonic motor 1, the rotator 5 and the first rotation shaft 7 have only to be configured to rotate simultaneously in the same direction in conjunction with each other.

The biasing member arranging region 7c is a region where the biasing member 10 is disposed. The biasing member arranging region 7c is a middle region between the rotator arranging region 7b and the support board member 11 (to be described later). Therefore, the biasing member 10 is arranged between the rotator 5 and the support board member 11.

The biasing member 10 is a constituent member configured to generate a biasing force for pressing the rotator 5, with the rotator 5 contacting a predetermined part on the surface of the elastic body 4 of the transducer 2. As the biasing member 10, a biasing spring in a coil shape is applied, for example. The biasing member 10 is arranged in the biasing member arranging region 7c of the first rotation shaft 7 of the output shaft 6, in the state of being wound coaxially with the first rotation shaft 7. Note that, as shown in FIG. 2, a shaft diameter D7 of the first shaft end region 7a is set to be smaller than a shaft diameter D5 of the biasing member arranging region 7c (D5>D7).

In this case, the biasing member 10 is in a power storing state, although the detail will be described later. In the power storing state, a first end 10a on the distal end side of the biasing member 10 is in contact with a first surface 5a of the rotator 5. In this state, the biasing member 10 presses the rotator 5 toward the distal end side in a direction along the Z-axis of the output shaft 6.

Here, the coupling configuration of the first rotation shaft 7 and the second rotation shaft 8 will be detailed.

The second rotation shaft 8 is coupled to the proximal end side of the first rotation shaft 7 by using the coupling pin 9. On the proximal end side of the first rotation shaft 7, a through hole 7d is formed. The through hole 7d is formed in a direction orthogonal to the axial direction of the first rotation shaft 7. The through hole 7d is formed in a long hole shape having a long side in the axial direction.

The one end of the second rotation shaft 8 is formed in a channel shape, with one side being open in the axial direction in the cross section as shown in FIG. 2. The opening diameter of the open end, which is oriented toward the axial direction, of the channel-shape portion of the second rotation shaft 8 is set to be slightly larger than the shaft diameter D7 of the first rotation shaft 7. Therefore, the proximal end side of the first rotation shaft 7 is insertable into and retractable from the open end of the second rotation shaft 8. The channel-shape portion of the second rotation shaft 8 includes a through hole 8d which is orthogonal to the axial direction.

The first rotation shaft 7 and the second rotation shaft 8, which are thus configured, are coupled with each other by using the coupling pin 9, to configure the output shaft 6. In other words, as shown in FIG. 2, firstly, the biasing member 10 is arranged in the biasing member arranging region 7c of the first rotation shaft 7. At this time, the first end 10a of the biasing member 10 is in contact with the first surface 5a of the rotator 5. In this state, the proximal end side of the first rotation shaft 7 is inserted, in the axial direction, into the open end of the channel-shape portion of the second rotation shaft 8. Then, a second end 10b of the biasing member 10 is brought into contact with the distal end surface of the second rotation shaft 8. This causes the biasing member 10 to be compressed by a predetermined amount between the rotator 5 and the second rotation shaft 8, to be brought into the power storing state.

Next, in this state, the through hole 7d and the through hole 8d are made to coincide with each other. Then, the coupling pin 9 is fitted into the through hole 7d and the through hole 8d. This causes the first rotation shaft 7 and the second rotation shaft 8 to be coupled coaxially with each other. At this time, the biasing member 10 is arranged in the compressed state between the rotator 5 and the second rotation shaft 8. In other words, the first end 10a on the distal end side of the biasing member 10 is in contact with the first surface 5a of the rotator 5 and the second end 10b on the proximal end side of the biasing member 10 is in contact with the distal end surface 8b of the second rotation shaft 8. This causes the biasing member 10 to be in the power storing state.

In this state, both shafts (the first rotation shaft 7 and the second rotation shaft 8) constitute the one output shaft 6. Therefore, both shafts (7, 8) in this state simultaneously rotate around the axis, in the same direction in conjunction with each other.

Furthermore, in this state, the first rotation shaft 7 and the second rotation shaft 8 are relatively movable in the axial direction by a predetermined distance. In this case, the relative movement of the first rotation shaft 7 and the second rotation shaft 8 in the axial direction is set to be within a length of the long side of the through hole 7d (the length denoted by the reference sign L1 in FIG. 2).

When the output shaft 6, which has been assembled as described above, is in a normal state, the biasing force of the biasing member 10 acts between the rotator 5 and the second rotation shaft 8. In other words, the biasing force acts to separate the both (5, 8) from each other in the axial direction.

Thus, the reason why the first rotation shaft 7 and the second rotation shaft 8 are configured to be movable relatively in the axial direction is to constantly ensure the state where the rotator 5 is pressed by the biasing force of the biasing member 10, to thereby cause the rotator 5 to be in contact with the predetermined part (operating point 7x to be described later, see FIG. 2) of the surface of the elastic body 4 of the transducer 2, in the state where the ultrasonic motor 1 is assembled.

On the other hand, on the proximal end side of the second rotation shaft 8 (the second shaft end of the output shaft 6), a protruding portion 8a in a short shaft shape is formed in the axial direction. The distal end of the protruding portion 8a is formed in substantially a protruded spherical shape. In addition, the protruding portion 8a is provided, with the rotation axis of the second rotation shaft 8 (output shaft 6) as a center.

Furthermore, the support board member 11 is disposed in the hollow portion 4a of the elastic body 4. Specifically, the support board member 11 is provided in a region which is closer to the proximal end of the second region 4n and which is the part having the inner diameter D8 in the hollow portion 4a. The support board member 11 is a plate-shaped member formed in substantially a disk shape having a diameter smaller than the inner diameter D8 of the hollow portion 4a. In FIG. 2, the outer diameter of the support board member 11 is denoted by the reference sign D9 (D8>D9).

Note that a space through which the power supply cable 13 is passed is formed in a space created between the outer circumference of the support board member 11 and the inner circumference of the hollow portion 4a.

The support board member 11 is fixed in the hollow portion 4a so as to be parallel to the surface orthogonal to the Z-axis direction. In addition, a recessed portion 11a having substantially a recessed spherical shape is formed at substantially the center part on the front face side of the support board member 11. When the ultrasonic motor 1 is assembled, the protruding portion 8a of the second rotation shaft 8 of the output shaft 6 is engaged with the recessed portion 11a, to thereby rotatably support the output shaft 6.

In such a configuration, when the ultrasonic motor 1 is in the assembled state, the output shaft 6 is arranged to pass through the hollow portion 4a of the elastic body 4 in the Z-axis direction. At this time, the first shaft end of the output shaft 6 (the first shaft end region 7a of the first rotation shaft 7) is arranged to protrude from the through hole 4c of the elastic body 4 to the outside of the elastic body 4. In addition, the second shaft end of the output shaft 6 (the protruding portion 8a of the second rotation shaft 8) is rotatably supported by the recessed portion 11a of the support board member 11.

At this time, the rotator 5 is pressed against the tapered surface 4d (inner wall surface) of the hollow portion 4a of the elastic body 4 by the biasing member 10. The rotator 5 is configured to rotate in this state. In detail, the first end 10a of the biasing member 10 is in contact with the first surface 5a of the rotator 5, and the biasing member 10 presses the rotator 5 toward the distal end side in the Z-axis direction. This causes a part of the second surface 5b of the rotator 5 to be pressed against the tapered surface 4d of the elastic body 4. The contacting part between the second surface 5b of the rotator 5 and the tapered surface 4d of the elastic body 4 in this state is illustrated as the operating point 7x in FIG. 2.

The position of the operating point 7x in the radial direction is preferably set at a position away from the axial center of the output shaft 6. This is because the further the position of the operating point 7x in the radial direction from the axial center of the output shaft 6 is set, the greater the rotation torque can be when the rotator 5 rotates.

Thus, when the rotator 5 receives the vibration of the transducer 2, with the rotator 5 contacting the tapered surface 4d of the elastic body 4 and being pressed by the biasing member 10, the vibration is transmitted to the rotator 5. In such a configuration, the frictional force generated between the rotator 5 and the tapered surface 4d causes the rotator 5 and the first rotation shaft 7 (output shaft 6) to rotate.

Therefore, in the contacting part (operating point 7x) at which the surface of the second surface 5b of the rotator 5 and the surface of the tapered surface 4d contact each other, both of the surfaces are preferably formed to have a predetermined surface roughness.

When the rotator 5 is pressed against the tapered surface 4d by the biasing member 10, the first shaft end region 7a of the first rotation shaft 7 is stably arranged at the axial center of the through hole 4c. Therefore, in this case, the tapered surface 4d is preferably formed to be smooth to some extent in the direction along the inclination of the tapered shape. In contrast, the tapered surface 4d is formed to have a predetermined surface roughness in the circumferential direction (rotation direction) thereof, so as to be capable of ensuring a necessary frictional force.

In addition, as shown in FIG. 2, the relationship shown below is set where:
in the hollow portion 4a of the elastic body 4, the inner diameter of the part in which the rotator 5 is disposed is D1;
the outer diameter of the rotator 5 is D2;
the outer diameter of the biasing member 10 is D3;
the inner diameter of the biasing member 10 is D4;
the outer diameter of the biasing member arranging region 7c of the output shaft 6 is D5;
the inner diameter of the through hole 4c of the elastic body 4 is D6; and
the outer diameter of at least the part of the first shaft end region 7a of the output shaft 6 is D7, the part protruding from the through hole 4c to the outside.

In other words, the following relationship,
hollow portion inner diameter D1>rotator outer diameter D2>through hole inner diameter D6
   is set.

In addition, the following relationships,
rotator outer diameter D2>biasing member inner diameter D4>biasing member arranging region outer diameter D5, and
hollow portion inner diameter D1>biasing member outer diameter D3 are set.

Furthermore, the following relationship,
through hole inner diameter D6>output shaft first shaft end outer diameter D7
   is set.

Brief description will be made below on the operation of the ultrasonic motor 1 according to the present embodiment thus configured. First, the alternating voltage, with the phase difference being 90 degrees and the driving frequency being the resonant frequency of the elastic body 4, is applied to each of the four piezoelectric elements 3, for example. This causes the elastic body 4 to be excited.

Then, the first region 4m of the elastic body 4, that is, the surface of the elastic body 4, which is the surface of the part where the piezoelectric elements 3 are pasted, generates vibration which is substantially a circular motion when viewed in the cross section.

At this time, the rotator 5 formed integrally with the first rotation shaft 7 of the output shaft 6 is pressed against the tapered surface 4d of the hollow portion 4a of the elastic body 4 by the biasing force of the biasing member 10. Therefore, the vibration generated in the first region 4m of the elastic body 4 is transmitted from the tapered surface 4d to the rotator 5. Then, the frictional force generated between the rotator 5 and the tapered surface 4d causes the rotator 5 to rotate around the Z axis. This causes the output shaft 6 also to be driven to rotate in the same direction as the rotator 5.

As described above, the ultrasonic motor 1 of the present embodiment includes the transducer 2 that vibrates when the alternating voltage is applied to the piezoelectric elements 3. The transducer 2 includes the piezoelectric elements 3 and the elastic body 4 to which the piezoelectric elements 3 are secured. In addition, the ultrasonic motor 1 includes the rotator 5 that rotates by receiving the vibration of the transducer 2 by the rotator 5 being pressed in a state of contacting the surface of the transducer 2. The ultrasonic motor 1 includes the biasing member 10 that presses the rotator 5 against the elastic body 4. The ultrasonic motor 1 includes the output shaft 6 that outputs the rotation of the rotator 5.

The elastic body 4 is formed in a pillar shape having the hollow portion 4a. The elastic body 4 houses the rotator 5 and the biasing member 10 in the hollow portion 4a. In addition, the rotator 5 rotates in the state of being pressed against the inner wall surface of the hollow portion 4a of the elastic body 4 by the biasing member 10.

Furthermore, the elastic body 4 includes, at the first end (distal end) thereof, the through hole 4c through which the first shaft end (7a) of the output shaft 6 rotatably penetrates. The elastic body 4 includes, in the hollow portion 4a, the support board member 11 that rotatably supports the second shaft end (8a) of the output shaft 6.

The output shaft 6 is arranged to pass through the hollow portion 4a. The first shaft end (7a) of the output shaft 6 is rotatably arranged so as to protrude from the through hole 4c to the outside of the elastic body 4. The second shaft end (8a) of the output shaft 6 is rotatably supported by the support board member 11.

The rotator 5 has substantially the disk shape. The rotator 5 is formed integrally and coaxially with the output shaft 6. The biasing member 10 is coaxial with the output shaft 6, and arranged in the power storing state between the rotator 5 and the support board member 11. The first end 10a of the biasing member 10 presses the first surface 5a of the rotator 5 in the axial direction along the output shaft 6.

The ultrasonic motor 1 thus configured according to the present embodiment is an actuator configured to convert the rotational force of the rotator 5 into a driving force for rotating the output shaft 6. To this end, the ultrasonic motor 1 generates characteristic vibration (resonance) in the elastic body 4 by applying the alternating voltage to the plurality of piezoelectric elements 3 provided on the outer surface side of the elastic body 4 in the state where the rotator 5 (rotor) which is provided in the hollow portion 4a of the elastic body 4 and which is pressurized by the biasing member 10 is made to contact the inner wall surface (tapered surface 4d) of the elastic body 4, to thereby transmit the vibration of the elastic body 4 to the rotator 5 by using the frictional force generated between the elastic body 4 and the rotator 5. Thereby, the rotational force of the rotator 5 causes the output shaft 6 to rotate.

With such a configuration, according to the ultrasonic motor 1 of the present embodiment, the rotator 5 is configured integrally with the output shaft 6, and the constituent members including the rotator 5, the output shaft 6 (7, 8, 9), the biasing member 10, the support board member 11, and the like are housed in the internal region of the elastic body 4, which can achieve the miniaturization.

Thus, the length of the elastic body 4 in the axial direction can be ensured while achieving the miniaturization of the ultrasonic motor 1, to thereby be capable of maintaining a low resonant frequency. As a result, the small-sized ultrasonic motor 1 capable of obtaining a sufficient rotation torque can be realized.

In addition, the contacting part (operating point 7x) between the elastic body 4 and the rotator 5 is provided in the hollow portion 4a of the transducer 2 (elastic body 4), to thereby be capable of reducing the number of the constituent elements to be exposed outside the ultrasonic motor 1. Accordingly, the ultrasonic motor 1 of the present embodiment can be easily incorporated into a small-sized apparatus such as an endoscope, for example.

In addition, the outer diameter D2 of the rotator 5 is set to be larger than the inner diameter D6 of the through hole 4c (D2>D6), which prevents the rotator 5 from falling off the through hole 4c to the outside. Simultaneously, the outer diameter D2 of the rotator 5 is set to be smaller than the inner diameter D1 of the hollow portion 4a of the elastic body 4 (D1>D2), which prevents the outer edge portion and the like of the rotator 5 from contacting the inner wall surface and the like of the internal region of the elastic body 4. Therefore, the rotation loss of the rotator 5 can be suppressed, and smooth rotation of the output shaft 6 can be ensured (D1>D2>D6).

Furthermore, the inner diameter D4 of the biasing member 10 is set to be smaller than the outer diameter D2 of the rotator 5 (D2>D4), which enables the pressing of the first surface 5a of the rotator 5 by the biasing member 10 to be surely maintained. Simultaneously, the inner diameter D4 of the biasing member 10 is formed to be larger than the outer diameter D5 of the middle region (7c) of the output shaft 6 (D4>D5), which enables the biasing member 10 to be arranged wound around the outer circumferential surface of the biasing member arranging region 7c of the first rotation shaft 7 of the output shaft 6, without the expansion and contraction thereof being interfered with. Furthermore, the outer diameter D3 of the biasing member 10 is formed to be smaller than the inner diameter D1 of the hollow portion 4a of the elastic body 4 (D1>D3), which can prevent the outer circumferential portion of the biasing member 10 from contacting the inner wall surface of the hollow portion 4a of the elastic body 4. Such a configuration can suppress the rotation loss of the rotator 5 and ensure the smooth rotation of the output shaft 6.

The outer diameter D7 of the first shaft end of the output shaft 6 (the first shaft end region 7a of the first rotation shaft 7) is formed to be smaller than the inner diameter D6 of the through hole 4c, which enables the first shaft end region 7a of the first rotation shaft 7 to be rotatably inserted into the through hole 4c.

In the hollow portion 4a of the elastic body 4, the inner wall surface with which the rotator 5 is brought into contact is formed as the tapered surface 4d, and the tapered surface 4d is formed toward the through hole 4c of the elastic body 4, which enables the vibration of the elastic body 4 to be transmitted efficiently in the axial direction of the rotator 5 and the output shaft 6.

The biasing member 10 is configured to press the first surface 5a of the rotator 5 in the axial direction of the output shaft 6, with the second surface 5b of the rotator 5 being made to contact the tapered surface 4d of the elastic body 4, which enables the axial center of the first rotation shaft 7 with which the rotator 5 is integrated to be arranged stably at the axial center of the through hole 4c. Such a configuration enables a stable rotation of the output shaft 6 to be ensured.

In the elastic body 4 or the rotator 5, the respective surfaces of the tapered surface 4d and the second surface 5b of the rotator 5 contacting at the contacting part (operating point 7x) have the predetermined surface roughness, to thereby be capable of surely holding the necessary sufficient frictional force.

The position of the contacting part (operating point 7x) at which the second surface 5b of the rotator 5 and the tapered surface 4d of the elastic body 4 contact each other in the radial direction is set at the position away from the axial center of the rotator 5 (output shaft 6) by the predetermined distance in the radial direction, which enables the necessary sufficient rotation torque to be obtained.

The surface roughness of the tapered surface 4d is set such that the surface roughness in the circumferential direction is larger than the surface roughness in the direction along the inclination, to thereby provide the stability of the axial center of the output shaft 6 by the rotator 5 being pressed by the biasing member 10, and at the same time, obtain a sufficient rotation torque by securing the frictional force between the rotator 5 and the elastic body 4.

Note that, in the configuration of the present embodiment, the elastic body 4 is shown by the example in which the elastic body 4 has substantially the pillar shape including the first region 4m, the second region 4n, and the hollow portion 4a. The elastic body 4 has only to be configured such that the piezoelectric elements 3 are secured on the outer surface thereof, and includes (a part of) the hollow portion 4a in which the rotator 5 and the biasing member 10 are housed. Therefore, the elastic body 4 is not limited to the configuration example shown in the above-described first embodiment. For example, the elastic body 4 may be formed in at least the part where the piezoelectric elements 3 are pasted of the part shown as the first region 4m, and the part shown as the second region 4n may be configured of a separated constituent member. In this case, the separated constituent member disposed in the part of the second region 4n may be made of a metal material or the like, and has only to be formed in substantially a pillar shape having a hollow portion, for example.

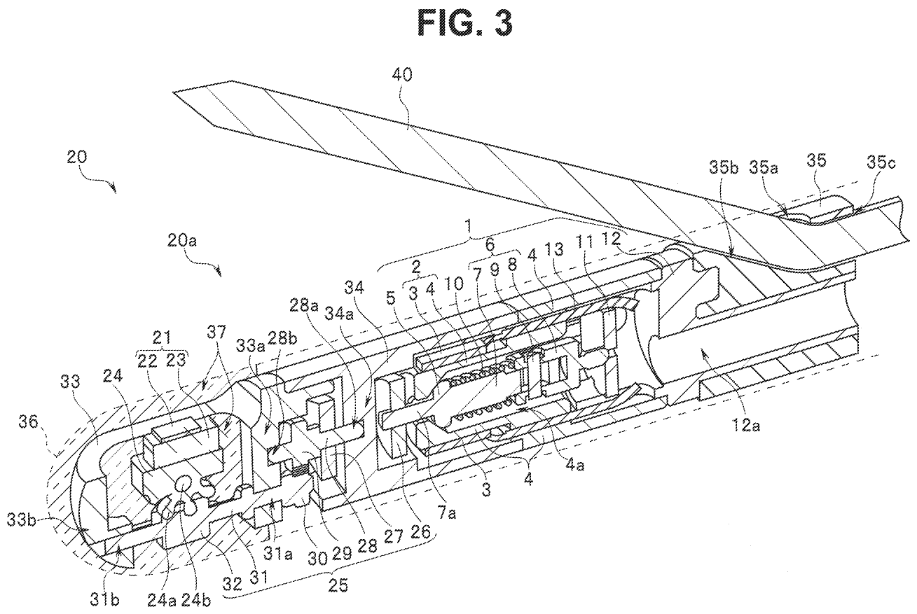

Next, description will be made on a configuration of an ultrasonic probe according to a second embodiment of the present invention with reference mainly to FIG. 3. FIG. 3 is a cross-sectional perspective view showing a main part of an internal configuration of a distal end portion of an ultrasonic probe according to the second embodiment of the present invention. Note that, in FIG. 3, the arrow signs X, Y, Z are defined similarly as in the above-described FIG. 1 and FIG. 2. Therefore, also in FIG. 3, the direction shown by the arrow sign Z is defined as the axial direction of the ultrasonic probe of the present embodiment and referred to as the Z-axis direction.

The ultrasonic probe of the present embodiment is an ultrasonic probe to which the ultrasonic motor 1 of the above-described first embodiment is applied. Since the configuration of the ultrasonic motor itself has been already described above, reference should be made to FIGS. 1 and 2, and detailed description will be omitted in the following description. In addition, the basic configuration of the ultrasonic probe of the present embodiment is substantially the same as that of conventional ultrasonic probes which are widely spread. Therefore, the configuration of the ultrasonic probe will be only described briefly in the following description.

An ultrasonic probe 20 of the present embodiment is suitable for insertion into a living body and is formed in an elongated tubular shape as a whole. The ultrasonic probe 20 is an ultrasonic probe that employs the mechanical scanning system, for example.

The ultrasonic probe 20 of the present embodiment includes, in a distal end portion 20a thereof, the above-described ultrasonic motor 1 (2 to 13), a transducer for ultrasonic image generation 21 (22, 23), a transducer oscillating base 24, an oscillation driving force transmitting mechanism 25 (26 to 32), a transducer holding member 33, an oscillation driving force transmitting portion holding member 34, an insertion portion housing 35, an outer sheath (resin tube) 36, an ultrasonic transmission medium 37, and the like.

The ultrasonic motor 1 (2 to 13) is the actuator described above in the first embodiment.

The transducer for ultrasonic image generation 21 (hereinafter simply referred to as the transducer 21) is constituted of a piezoelectric element for ultrasonic image generation 22 (hereinafter simply referred to as the piezoelectric element 22) and an elastic body for ultrasonic image generation 23 (hereinafter simply referred to as the elastic body 23). The transducer 21 is configured such that the piezoelectric element 22 is pasted on the elastic body 23. The transducer 21 vibrates when an alternating voltage is applied to the piezoelectric element 22.

The piezoelectric element 22 is an electromechanical conversion element. A power supply cable (not shown) is connected to the piezoelectric element 22. The elastic body 23 is made of a metal material, and configured to receive vibration of the piezoelectric element 22 to generate a characteristic vibration (resonance). The vibration is a vibration for ultrasonic image generation.

The transducer 21 is placed on and fixed to the transducer oscillating base 24. The transducer oscillating base 24 receives the rotation driving force transmitted from the ultrasonic motor 1 through the oscillation driving force transmitting mechanism 25 (to be detailed later), to cause the transducer 21 to oscillate in a predetermined direction within a predetermined range.

The transducer oscillating base 24 is provided rotatably around an oscillation shaft 24b with respect to a fixed portion (transducer holding member 33). In this case, the oscillation shaft 24b is arranged in a direction orthogonal to the Z-axis direction of the ultrasonic probe 20. Therefore, the transducer oscillating base 24 oscillates with respect to the Z-axis direction of the ultrasonic probe 20.

In addition, the transducer oscillating base 24 includes a helical gear 24a. The helical gear 24a is a worm wheel in a worm gear mechanism that converts the rotation driving force of the ultrasonic motor 1, which is transmitted through the oscillation driving force transmitting mechanism 25 (to be detailed later), into the rotation driving force around the oscillation shaft 24b. The worm (screw gear) corresponding to the helical gear 24a is a worm portion 32 (to be detailed later) included in the oscillation driving force transmitting mechanism 25.

With such a configuration, the transducer oscillating base 24 oscillates in a predetermined direction within a predetermined rotation angle according to a rotation direction and a rotation angle of the worm portion 32 of the oscillation driving force transmitting mechanism 25.

The oscillation driving force transmitting mechanism 25 transmits the rotation driving force outputted from the output shaft 6 (the first shaft end region 7a of the first rotation shaft 7) of the ultrasonic motor 1 to the transducer oscillating base 24.

The oscillation driving force transmitting mechanism 25 includes a first magnet 26, a second magnet 27, a first shaft 28, a first gear 29, a second gear 30, a second shaft 31, and the worm portion 32.

The first magnet 26 is a magnet formed in substantially a ring shape in which an N-pole and an S-pole are alternately arranged in the circumferential direction. The first magnet 26 is fixed coaxially to the first rotation shaft 7 of the output shaft 6 of the ultrasonic motor 1. In more detail, the first magnet 26 is fixed to a shaft part of the first shaft end region 7a of the first rotation shaft 7, the shaft part protruding outside from the elastic body 4. With such a configuration, the first magnet 26 rotates in the same direction as the first rotation shaft 7, in accordance with the rotation of the first rotation shaft 7.

The second magnet 27 is formed in substantially the same shape as the first magnet 26. The second magnet 27 is fixed coaxially to the first shaft 28. The first magnet 26 and the second magnet 27 are arranged, with the respective plate surfaces thereof being opposed to each other, separated by a partition wall 34b of the oscillation driving force transmitting portion holding member 34 (to be detailed later) therebetween. Such a configuration provides, what is called, a non-contact magnetic joint (magnetic coupling), in which, when the first magnet 26 rotates to cause the respective magnetic poles to move in the rotation direction, a magnetic force acts on the respective magnetic poles of the second magnet 27 arranged opposed to the first magnet 26, to thereby cause the second magnet 27 to rotate in the non-contact manner.

The first shaft 28 is a shaft member configured to fix the above-described second magnet 27 and the first gear 29 coaxially. A proximal end 28a of the first shaft 28 is rotatably supported by the partition wall 34a of the oscillation driving force transmitting portion holding member 34. In addition, a distal end 28b of the first shaft 28 is rotatably supported by a partition wall 33a of the transducer holding member 33. The first shaft 28 is arranged in a manner that the axial center thereof substantially coincides with the axial center of the output shaft 6 (first rotation shaft 7) of the ultrasonic motor 1 in the Z-axis direction. The first shaft 28 rotates in the same direction as the second magnet 27 when the second magnet 27 rotates by the above-described operation.

The first gear 29 is a spur gear fixed coaxially to the first shaft 28. When the second magnet 27 rotates by the above-described operation to thereby cause the first shaft 28 to rotate, the first gear 29 rotates in the same direction.

The second gear 30 is a spur gear configured to be meshed with the first gear 29. In accordance with the rotation of the first gear 29, the second gear 30 rotates in the direction opposite to the rotation direction of the first gear 29. The second gear 30 is fixed coaxially to the second shaft 31 at the proximal end portion of the second shaft 31. With such a configuration, when the second gear 30 rotates in accordance with the rotation of the first gear 29, the second shaft 31 rotates in the same direction as the second gear 30.

The second shaft 31 is a shaft member that fixes the above-described second gear 30 and the worm portion 32 coaxially. A middle portion 31a of the second shaft 31, which is closer to the proximal end of the second shaft 31, is rotatably arranged penetrating the partition wall 33a of the transducer holding member 33. In addition, a distal end 31b of the second shaft 31 is rotatably supported by the distal end wall 33b of the transducer holding member 33. Then, the second shaft 31 is arranged such that the axial center thereof is substantially parallel with (that is, in the Z-axis direction) the axial center of the output shaft 6 (first rotation shaft 7) of the ultrasonic motor 1. In accordance with the rotation of the second gear 30, the second shaft 31 rotates in the same direction as the second gear 30.

The worm portion 32 is a screw gear fixed coaxially to the second shaft 31. The worm portion 32 is a worm portion (screw gear) in the worm gear mechanism that converts the rotation driving force of the ultrasonic motor 1 into the rotation driving force around the oscillation shaft 24b. The worm wheel corresponding to the worm portion 32 is the above-described helical gear 24a. As described above, when the second shaft 31 rotates, the worm portion 32 rotates in the same direction as the second shaft 31. With such a configuration, the worm portion 32 causes the transducer oscillating base 24 to oscillate in a predetermined direction through the helical gear 24a.

The transducer holding member 33 is a protection member which houses, inside thereof, the transducer oscillating base 24 to which the transducer 21 is fixed, and the worm gear mechanism part of the oscillation driving force transmitting mechanism 25, and which covers around these constituent members. The transducer holding member 33 is arranged at the most distal end side of the ultrasonic probe 20.

The oscillation driving force transmitting portion holding member 34 is a protection member which houses, inside thereof, the main constituent members (the constituent members other than the above-described worm gear mechanism) of the oscillation driving force transmitting mechanism 25 and the ultrasonic motor 1 as a driving source, and which covers around these constituent members. The oscillation driving force transmitting portion holding member 34 is coupled to the proximal end side of the transducer holding member 33. A part of the ultrasonic motor 1 (the second region 4n on the proximal end side of the elastic body 4) is connected to the proximal end side of the oscillation driving force transmitting portion holding member 34. In this case, another part of the ultrasonic motor 1 (the first region 4m on the distal end side of the elastic body 4) is arranged inside the proximal end side of the oscillation driving force transmitting portion holding member 34.

The insertion portion housing 35 includes an insertion pathway (not shown) through which a power supply cable 13 and other signal cables, etc., (not shown) extended from the respective constituent members in the distal end portion 20a of the ultrasonic probe 20 are passed, and also includes an insertion path 35c of a puncture needle 40. The insertion portion housing 35 is coupled to a rear cylindrical member 12 of the ultrasonic motor 1.

Note that the insertion portion housing 35 includes, inside thereof, the insertion path 35c of the puncture needle 40. At the distal end part of the insertion portion housing 35, an opening 35a of the insertion path 35c and a puncture needle raising base 35b are formed.

The opening 35a is an opening through which the distal end portion of the puncture needle 40 passed through the insertion path 35c is protruded to the outside. The insertion path 35c is a passage through which the puncture needle 40 is passed. The puncture needle raising base 35b is a constituent part for defining the protruding direction of the puncture needle 40 when the distal end portion of the puncture needle 40 passed through the insertion path 35c is protruded to the outside. The puncture needle raising base 35b is formed with an inclined surface having a predetermined angle with respect to the Z-axis direction of the ultrasonic probe 20. With such a configuration, when the puncture needle 40 is pushed out through the insertion path 35c, the distal end of the puncture needle 40 is guided by the puncture needle raising base 35b and the direction of the distal end of the puncture needle is changed toward the opening 35a to be protruded to the outside of the ultrasonic probe 20.

The puncture needle 40 is an elongated hollow flexible tubular member, the distal end of which is formed in a needle shape. The puncture needle 40 is configured to be able to be inserted from the insertion port (not shown) on the operation portion side to be passed through the insertion path 35c in the ultrasonic probe 20, and then protruded from the opening 35a of the distal end portion 20a of the ultrasonic probe 20.

Note that the transducer holding member 33, the oscillation driving force transmitting portion holding member 34, and the insertion portion housing 35 are formed such that the outer surfaces thereof are substantially flush with one another, and the ultrasonic probe 20 is formed in an elongated tubular shape as a whole. In this case, as described above, a part (the second region 4n) of the elastic body 4 of the ultrasonic motor 1 is disposed between the oscillation driving force transmitting portion holding member 34 and the insertion portion housing 35. The outer surface of the second region 4n of the elastic body 4 is also formed to be substantially flush with the outer surfaces of the above-described respective members (33, 34, 35).

The respective outer surfaces of the transducer holding member 33, the oscillation driving force transmitting portion holding member 34, a part (the second region 4n of the elastic body 4) of the ultrasonic motor 1, and the insertion portion housing 35 are covered with an outer sheath 36. As the outer sheath 36, a tubular member made of resin is applied, for example. Note that the outer sheath 36 is simplified to be shown by dotted lines in FIG. 3, in order to avoid the complication of the drawing.

In the distal end of the outer sheath 36, inside the outer sheath 36 and the internal space of the transducer holding member 33 are filled with an ultrasonic transmission medium 37. The ultrasonic transmission medium 37 is a medium for suppressing attenuation of the ultrasound outputted from the transducer 21. As the ultrasonic transmission medium 37, for example, a liquid oil, a jelly oil, and the like are applied.

Hereinafter, brief description will be made on the operation of the ultrasonic probe 20 thus configured according to the present embodiment.

First, when the ultrasonic motor 1 is driven as described above, the output shaft 6 rotates. When the output shaft 6 rotates, the first rotation shaft 7 rotates, and also the first magnet 26 fixed coaxially to the first rotation shaft 7 rotates in the same direction as the first rotation shaft 7. When the first magnet 26 rotates, the second magnet 27 rotates. Following this, the rotation driving force outputted from the ultrasonic motor 1 is transmitted to the first shaft 28, the first gear 29, the second gear 30, the second shaft 31, and the worm portion 32. Then, the worm portion 32 causes the transducer oscillating base 24 to oscillate. Thereby, the transducer 2 oscillates.

Thus, the rotation driving force outputted from the ultrasonic motor 1 is transmitted to the transducer 2 through the oscillation driving force transmitting mechanism 25. At this time, the rotation direction, the rotation speed, and the like of the ultrasonic motor 1 are controlled at a predetermined timing appropriately, to thereby enable the transducer 2 to oscillate within a predetermined range. This enables the oscillating scan by the transducer 2 to be achieved, to thereby enable an ultrasonic image in a predetermined range to be generated.

Then, an operator operates the ultrasonic probe 20 while observing the ultrasonic image displayed on a display apparatus (not shown), to search for a lesion part to be examined. When the operator found the lesion part to be examined, the operator performs puncturing operation on the lesion part to be examined by using the puncture needle 40.

According to the ultrasonic probe 20 of the present embodiment, only the operation for pushing the puncture needle 40 in the axial direction causes the puncture needle 40 to protrude from the opening 35a to the outside at a predetermined protruding angle with respect to the Z-axis direction by the puncture needle raising base 35b. Then, the lesion part to be examined is punctured with the puncture needle 40, to suck and collect living tissues, cells, or the like of the lesion part. The living tissues, cells, or the like thus collected are utilized for a pathological examination or a pathological diagnosis.

With such a configuration, according to the ultrasonic probe of the second embodiment, the ultrasonic motor 1 applied to the ultrasonic probe houses, in the internal region of the elastic body 4, the constituent members such as the rotator 5, the output shaft 6 (7, 8, 9), the biasing member 10, the support board member 11, and the like, and also includes, in the internal region of the elastic body 4, the contacting part (operating point 7x) between the rotator 5 and the elastic body 4. With such a configuration, the vicinity of the operating point 7x of the ultrasonic motor 1 is not exposed to the ultrasonic transmission medium 37 (a liquid oil, a jelly oil, or the like) provided around the transducer for ultrasonic image generation 21.

In addition, the partition wall 33a of the transducer holding member 33, the partition wall 34a of the oscillation driving force transmitting portion holding member 34, and the like are provided between the transducer 21 housed in the internal space of the transducer holding member 33 and the ultrasonic motor 1. Such a configuration prevents the ultrasonic transmission medium 37 in the internal space of the transducer holding member 33 from intruding into the region where the ultrasonic motor 1 is arranged. With this configuration, there is no concern for the intrusion of the ultrasonic transmission medium 37 into the internal region of the ultrasonic motor 1. Therefore, the frictional force generated at the operating point 7x in the ultrasonic motor 1 can be surely ensured, and the sufficient rotation torque of the ultrasonic motor 1 can be obtained easily.

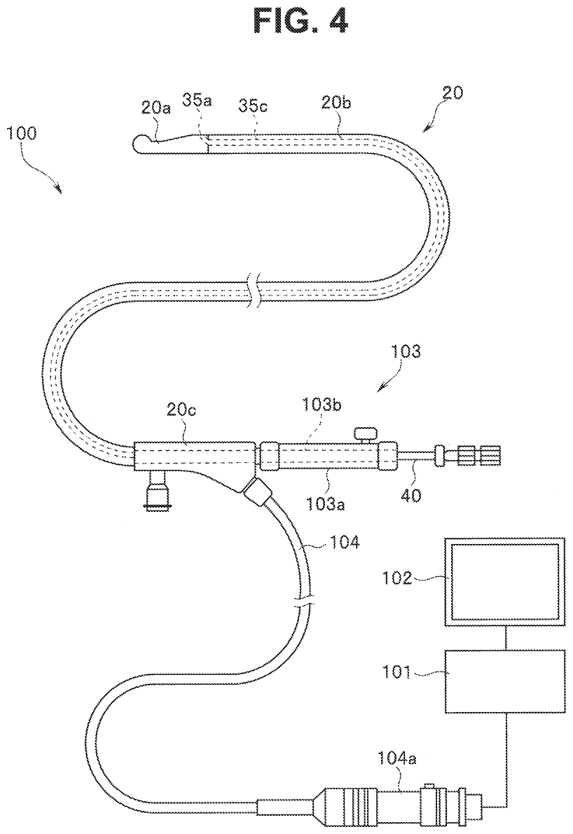

Next, description will be made below on the schematic configuration of an ultrasonic endoscope system according to a third embodiment of the present invention with reference to FIG. 4. FIG. 4 is a schematic configuration view schematically showing an overall configuration of the ultrasonic endoscope system according to the third embodiment of the present invention.

The ultrasonic endoscope system according to the present embodiment exemplifies an ultrasonically-guided puncture system including an endoscope which includes, at the distal end thereof, the ultrasonic probe according to the second embodiment to which the ultrasonic motor according to the first embodiment is applied. The ultrasonically-guided puncture system 100 generates an ultrasonic image in a body cavity of a living body which is an object to be examined, for example, a very thin lumen such as a blood vessel in a peripheral part of a lung, a prostate gland, and the like, by using the ultrasonic probe, and displays the ultrasonic image on a display apparatus, to thereby enable an operator to search for and observe a lesion part to be examined, and the ultrasonically-guided puncture system 100 has a function for enabling the operator to puncture the lesion part to be examined with a puncture needle to suck and collect living tissues, cells, and the like of the lesion part.

As shown in FIG. 4, the ultrasonically-guided puncture system 100 of the present embodiment includes an ultrasonic probe 20 having an elongated tubular shape, a puncture needle device 103, a processor 101, a monitor 102, and the like.

It is supposed that the same type of ultrasonic probe as that described in the above-described second embodiment is applied as the ultrasonic probe 20. As described above, the ultrasonic motor 1 of the first embodiment is applied to the ultrasonic probe 20. The ultrasonic probe 20 has the elongated tubular shape which can be inserted into a very thin lumen such as a blood vessel, for example, and is, what is called, a mechanical scanning type ultrasonic probe.

The ultrasonic probe 20 includes a distal end portion 20a and a flexible tube portion 20b. The distal end portion 20a houses a transducer, a transducer driving mechanism, and the like. The flexible tube portion 20b is elongated and flexible tube made of resin.

The ultrasonic probe 20 has a diameter of about 2 to 4 mm, for example. The distal end portion 20a houses, inside thereof, not only a transducer for ultrasonic image generation 21, but also an oscillation driving force transmitting mechanism 25 for driving the transducer 21, the ultrasonic motor 1 as a driving source, and the like. Note that, with regard to the detailed configuration of the ultrasonic probe 20, reference should be made to FIG. 3, and the detailed description thereof will be omitted. Furthermore, with regard to the detailed configuration of the ultrasonic motor 1, reference should be made to FIGS. 1, and 2, and the detailed description thereof will be omitted.

At the proximal end of the flexible tube portion 20b of the ultrasonic probe 20, a puncture needle device connector 20c is provided. The puncture needle device connector 20c is a connecting member provided for connecting the ultrasonic probe 20 and a puncture needle device 103.

In addition, the ultrasonic probe 20 includes, inside thereof, an insertion path 35c through which a puncture needle 40 is passed in the longitudinal axis direction (see also FIG. 3). The insertion path 35c leads to an opening 35a formed on an insertion portion housing 35 in the distal end portion 20a.

Furthermore, the ultrasonic probe 20 includes, inside thereof, an insertion pathway (not shown) through which the power supply cable 13 (see FIG. 3) and a plurality of other signal cables, etc., (not shown) extended from the respective constituent members in the distal end portion 20a are passed. These plurality of signal cables transmit, for example, a control signal and the like for driving and controlling the ultrasonic motor 1.

A cable 20d is extended from the puncture needle device connector 20c. A connector 20e is provided at the distal end of the cable 20d. The connector 20e is a connector for connecting the ultrasonic probe 20 and the processor 101 through the cable 20d.

The puncture needle device 103 includes the puncture needle 40, a puncturing handle 103a for operating the puncture needle 40.

The puncturing handle 103a is coupled to the puncture needle device connector 20c. The puncturing handle 103a includes a puncture needle insertion channel 103b communicating with the insertion path 35c of the ultrasonic probe 20 through the inside of the puncture needle device connector 20c.

When the puncture needle 40 is inserted into the puncture needle insertion channel 103b, the puncture needle 40 is introduced into the insertion path 35c of the ultrasonic probe 20, and finally, the distal end of the puncture needle 40 is protruded outside from the opening 35a of the distal end portion 20a of the ultrasonic probe 20.

The processor 101 includes: a driving control section configured to drive the transducer for ultrasonic image generation, the ultrasonic motor, and the like in the distal end portion 20a of the ultrasonic probe 20; an image generation section configured to generate an ultrasonic image around the lesion part to be examined, based on the output signal from the transducer for ultrasonic image generation; and the like.

The monitor 102 is a display apparatus that receives an ultrasonic image signal generated by the processor 101, performs image processing for converting the received ultrasonic image signal into a signal suitable for display, and displays an ultrasonic image.

The processor 101 and the monitor 102 are connected to each other by a predetermined signal cable. In addition, a power cable for receiving power supply from a commercial power source, for example, is connected to each of the processor 101 and the monitor 102, although the illustration thereof is omitted.

Note that the processor 101 includes hardware, and includes, for example, a central processing unit (CPU), a ROM, a RAM, and the like. The CPU reads a software program stored in the ROM, expands the program in the RAM, and executes the program, to thereby realize the functions of the driving control section, the image generation section, and the like. The processor may be configured of a semiconductor chip such as an FPGA (Field Programmable Gate Array), for example. Furthermore, the driving control section, the image generation section, etc., may be configured by electronic circuits.

With the ultrasonic endoscope system 100 thus configured, a user can perform a series of operations including observing the ultrasonic image displayed on the monitor 102, operating the puncture needle 40 while observing the displayed image, performing puncturing operation into the lesion part to be examined, and sucking and collecting the living tissues, cells, and the like of the lesion part. The living tissues, cells, and the like thus collected are utilized for a pathological examination or a pathological diagnosis.

The present invention is not limited to the above-described embodiments, and it goes without saying that various modifications and applications can be implemented within a range without departing from the subject matter of the invention. Furthermore, the above embodiments include inventions at various stages, and various inventions can be extracted by appropriate combinations of a plurality of disclosed constituent elements. For example, even when some of the constituent elements are removed from all the constituent elements shown in the above-described one embodiment, a configuration from which the constituent elements are eliminated can be extracted as an invention insofar as the configuration can solve the problem to be solved by the invention and attain the effects of the invention. Furthermore, constituent elements over different embodiments may be combined as appropriate. The invention is not limited by the specific embodiments except as limited by appended claims.

What is claimed is:

1. An ultrasonic motor comprising:
a transducer comprising a plurality of piezoelectric elements and an elastic body having an outer wall surface on which the plurality of piezoelectric elements are secured, the transducer being configured to vibrate when an alternating voltage is applied to the plurality of piezoelectric elements;
a rotator configured to rotate by receiving the vibration of the transducer by being pressed in contact with a surface of the transducer;
a biasing spring configured to press the rotator against the elastic body; and
an output shaft configured to output rotation of the rotator, wherein
the elastic body includes a hollow portion, and configured to house the rotator and the biasing spring in the hollow portion, and
the rotator rotates in a state of being pressed against an inner wall surface of the hollow portion of the elastic body by the biasing spring.

2. The ultrasonic motor according to claim 1, wherein
the elastic body includes, at a first end of the elastic body, a through hole through which a first shaft end of the output shaft rotatably penetrates, and includes, in the hollow portion, a support board member configured to rotatably support a second shaft end of the output shaft,
the output shaft is arranged to penetrate the hollow portion, the first shaft end protrudes from the through hole to an outside of the elastic body to be arranged rotatably, and the second shaft end is rotatably supported by the support board member,
the rotator has substantially a disk shape, and formed integrally and coaxially with the output shaft, and
the biasing spring is arranged in a compressed state in a middle region between the rotator and the support board member so as to be coaxial with the output shaft, and a first end of the biasing spring presses a first surface of the rotator in a direction along the output shaft.

3. The ultrasonic motor according to claim 2, wherein
the rotator has an outer diameter which is formed to be larger than an inner diameter of the through hole and smaller than an inner diameter of the hollow portion of the elastic body,
the biasing spring has an inner diameter which is formed to be smaller than the outer diameter of the rotator and larger than an outer diameter of the middle region of the output shaft, and the biasing spring has an outer diameter which is formed to be smaller than the inner diameter of the hollow portion of the elastic body, and
the first shaft end of the output shaft has an outer diameter which is formed to be smaller than the inner diameter of the through hole.

4. The ultrasonic motor according to claim 2, wherein the hollow portion of the elastic body includes a tapered surface formed toward the through hole.

5. The ultrasonic motor according to claim 4, wherein
the biasing spring presses the first surface of the rotator toward an axial direction of the output shaft, with a second surface of the rotator and the tapered surface of the elastic body being in contact with each other.

6. The ultrasonic motor according to claim 4, wherein
both of the tapered surface of the elastic body and the second surface of the rotator have a predetermined surface roughness at a contacting part at which the tapered surface and the second surface contact each other.

7. The ultrasonic motor according to claim 4, wherein
a position of the contacting part between the second surface of the rotator and the tapered surface of the elastic body in a radial direction is set to a position away from an axial center of the rotator in the radial direction by a predetermined distance.

8. An ultrasonic probe comprising:
the ultrasonic motor according to claim 2; and
an ultrasonic transducer configured to receive rotation of the output shaft, to transmit and receive an ultrasound.

9. An ultrasonic endoscope system comprising:
an endoscope including the ultrasonic probe according to claim 8 in a distal end of the endoscope;
a processor; and
a monitor, wherein
the ultrasonic probe is configured to transmit an ultrasound to a subject, and receive the ultrasound reflected by the subject,
the processor is configured to generate an image signal based on the ultrasound received by the ultrasonic probe, and
the monitor is configured to receive the image signal to display an image of the subject.
